(19) United States
(12) Patent Application Publication
(10) Pub. No.: US 2010/0221502 A1
(43) Pub. Date: Sep. 2, 2010

(54) Anthrapyridone Compound and Salt Thereof, Magenta Ink Composition Containing the Anthrapyridone Compound, and Colored Article

(76) Inventors: Hiroyuki Matsumoto, Tokyo (JP); Noriko Kajiura, Tokyo (JP)

Correspondence Address: Nields, Lemack & Frame, LLC, 176 E. Main Street, Suite #5, Westborough, MA, 01581, US

(21) Appl. No.: 11/989,983

(22) PCT Filed: Aug. 7, 2006
(86) PCT No.: PCT/JP2006/315592

§ 371 Date: Feb. 4, 2008

(30) Foreign Application Priority Data
Aug. 9, 2005 (JP) 2005-231215

(51) Int. Cl. C09B 5/14; C07D 403/12; C09D 11/02; B05D 5/06; B32B 9/00; B41J 2/175

(52) U.S. Cl. 428/195.1; 544/209; 544/212; 106/31.47; 427/256; 427/288; 428/704; 347/85; 347/100

(57) ABSTRACT

The present invention relates to a novel anthrapyridone compound represented by the following formula (1)

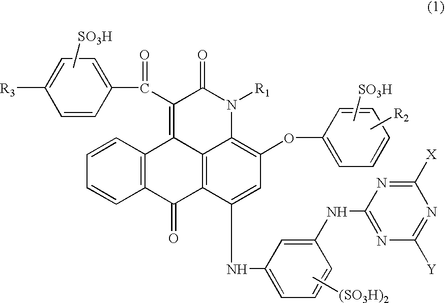

(wherein, R₁ represents a hydrogen atom, an alkyl group and the like, R₂ represents a hydrogen atom, an alkyl group, a phenyl group, a phenoxy group and the like, R₃ represents a hydrogen atom, or a methoxy group, X represents an anilino group which may have a substituent such as a sulfonic acid group; a naphthylamino group which may be substituted by a sulfonic acid group; a mono- or di-alkylamino group which may have a substituent such as a sulfonic acid group; an aralkylamino group; a cycloalkylamino group; a phenoxy group which may have a substituent such as a sulfonic acid; a hydroxy group; an amino group; and the like, Y represents a chlorine atom; a hydroxy group; an amino group; and the like)
in free acid form, an ink composition containing the same and a colored article using the same, and images recorded by an ink for inkjet recording containing said compound have high vividness and exhibit superior light fastness and ozone gas fastness.

### TECHNICAL FIELD

The present invention relates to a novel anthrapyridone compound, a magenta ink composition comprising the anthrapyridone compound and a colored article obtained by using the same.

### BACKGROUND ART

In the recording method by means of an ink jet printer which is one of the typical methods among various color recording methods, various methods for discharging ink have been developed, where ink droplets are generated and deposited onto various record-receiving materials (paper, film, cloth and the like) to perform recording. This has been rapidly prevailing lately and is expected to continue growing remarkably in the future because of such features as quietness with less noise generation due to no contact of a recording head with a record-receiving material and as easiness in downsizing, speedup and colorization.

Conventionally, as an ink for fountain pens or felt pens and an ink for inkjet recording, water-based inks dissolving a water-soluble dye in an aqueous medium have been used, and in these water-soluble inks, a water-soluble organic solvent is generally added to prevent ink from clogging at a pen tip or an inkjet nozzle. These conventional inks are required to provide recorded images with sufficient density, not to clog at a pen tip or an inkjet nozzle, to dry quickly on record-receiving materials, to bleed less, and to have good storage stability. In addition, images to be formed are required to have fastnesses such as water fastness, light fastness and moisture fastness.

Meanwhile, images or character information on color displays of computers are generally expressed by subtractive color mixing of 4 color inks of yellow (Y), magenta (M), cyan (C) and black (K) for color recording by an ink jet printer. In order to reproduce, as faithfully as possible, the hues of images expressed by additive color mixing of red (R), green (G) and blue (B) on CRT displays and the like, through images by subtractive color mixing, it is desired that each of Y, M and C has a hue as close to each standard as possible and vividness. In addition, it is required that an ink composition to be used is stable in storage for a long period of time, and that printed images have a high concentration and are excellent in fastnesses such as water fastness, light fastness and gas fastness.

The use of ink jet printers has been increased even in compact printers for OA to large scale printers for industries, and fastnesses such as water fastness, moisture fastness, light fastness and gas fastness are required for ink jet printers more than before. Water fastness has been considerably improving by coating the surface of paper with organic or inorganic fine particles, which can absorb coloring matter in inks, such as porous silica, cation polymer, aluminasol or special ceramic, together with a PVA resin and the like. Moisture fastness is durability against the phenomenon that dye coloring matter in record-receiving materials bleeds when colored record-receiving materials are stored under the atmosphere of high humidity. When bleeding of dye coloring matter occurs, the image quality is significantly reduced especially in images where the image quality of photo like high resolution is required, and it is important that such bleeding occurs as less as possible. Technique to considerably improve the light fastness has not established yet and also many magenta coloring matters are originally poor in light fastness among 4 primary colors of Y, M, C and K, so its improvement is an important problem. In addition, with the recent spread of digital cameras, chances to print photos at home are increasing, and discoloration of images by oxidizing gases in the air such as ozone gas and nitrogen oxides during storage of printed articles obtained is also regarded as a problem. Oxidizing gases have a property of reacting with dye on or in recorded paper leading to discoloration or fading of printed images. Ozone gas is regarded as a main causative substance among oxidizing gases to accelerate discoloration of inkjet recorded images. This discoloration or fading is a characteristic of inkjet images, and improvement of gas fastness is an important problem as well as improvement of light fastness.

As the skeleton of magenta coloring matter used for water-soluble inks for inkjet recording, typical is an azo coloring matter using H acid (1-amino-8-hydroxy-naphthalene-3,6-disulfonic acid) and xanthene coloring matter. However, the xanthene coloring matter has a far superior hue and vividness but has far inferior light fastness. Meanwhile, some azo coloring matters using H acid are good in terms of hue and water fastness, but have inferior light fastness, gas fastness and vividness. This type of magenta dye having superior vividness and light fastness is developed, but it still has an inferior level of light fastness compared with dye having another hue such as cyan dye as typified by copper phthalocyanine coloring matter or a yellow dye.

An anthrapyridone coloring matter (for example, see Patent Literatures 1 to 8) is a coloring matter having a skeleton for magenta superior in vividness and light fastness, but a coloring matter satisfying all the respects such as hue, vividness, light fastness, water fastness, gas fastness and solution stability has not been achieved yet.
[Patent Literature 1] JP S59-74173 A (Pages 1 to 3)
[Patent Literature 2] JP H2-16171 A (Pages 1 and 5 to 7)
[Patent Literature 3] JP 2000-109464 A (Pages 1 to 2 and 8 to 12)
[Patent Literature 4] JP 2000-169776 A (Pages 1 to 2 and 6 to 9)
[Patent Literature 5] JP 2000-191660 A (Pages 1 to 3 and 11 to 14)
[Patent Literature 6] JP 2001-72884 A (Pages 1 to 2 and 8 to 11)
[Patent Literature 7] JP 2001-139836 A (Pages 1 to 2 and 7 to 12)
[Patent Literature 8] JP 2003-192930 A (Pages 22 and 36 to 37)

### DISCLOSURE OF THE INVENTION

### Problems to Be Solved by the Invention

The object of the present invention is to provide a coloring matter (compound) for magenta which has high solubility to water, and hue and vividness suitable for inkjet recording, and enable recorded objects to have excellent light fastness, moisture fastness and gas fastness, and an ink composition containing it.

### Means of Solving the Problems

The inventors of the present invention intensively studied a way to solve the above problems and have found that an anthrapyridone compound represented by a specific formula can solve the above problems and completed the present invention.

That is, the present invention relates to
(1) An anthrapyridone compound represented by the following formula (1)

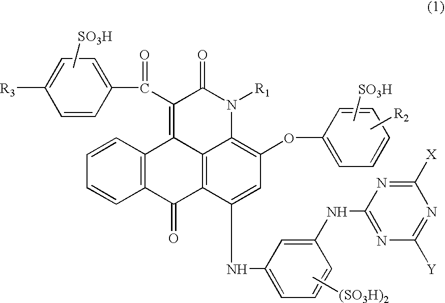

(wherein R₁ represents a hydrogen atom, an alkyl group, a hydroxy lower alkyl group, a cyclohexyl group, a mono- or di-alkylaminoalkyl group or a cyano lower alkyl group, R₂ represents a hydrogen atom, an alkyl group, a phenyl group, a sulfophenyl group, a phenoxy group or a sulfophenoxy group, R₃ represents a hydrogen atom or a methoxy group, X represents (1) an anilino group which may have a group selected from the group consisting of a sulfonic acid group, a carboxyl group, a methyl group, a methoxy group, an anilino group and a phenoxy group as a substituent; (2) a carboxy-hydroxyanilino group; (3) a naphthylamino group which may be substituted by a sulfonic acid group; (4) a mono- or di-alkylamino group which may have a group selected from the group consisting of a sulfonic acid group, a carboxyl group and a hydroxy group as a substituent; (5) an aralkylamino group; (6) a cycloalkylamino group; (7) a phenoxy group which may have a group selected from the group consisting of a sulfonic acid group, a carboxyl group, an acetylamino group, an amino group, a hydroxy group, a phenoxy group and a phenyl group as a substituent; (8) a monoalkylaminoalkylamino group; (9) a dialkylaminoalkylamino group; (10) a hydroxy group; or (11)an amino group, Y represents a chlorine atom; a hydroxy group; an amino group; a mono- or di-alkylamino group which may have a substituent selected from the group consisting of a sulfonic acid group, a carboxyl group and a hydroxy group on the alkyl group; or a morpholino group, respectively)
in free acid form,
(2) The anthrapyridone compound according to the above (1), which is represented by the following formula (2)

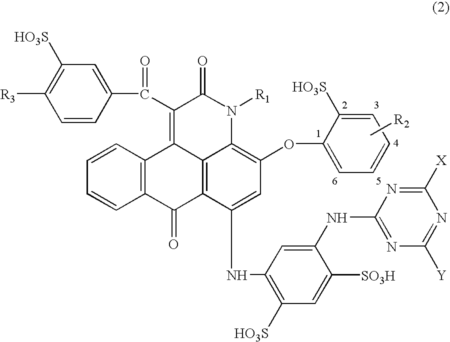

(wherein, R₁, R₂, R₃, X and Y have the same meanings as in the formula (1))
in free acid form,
(3) The anthrapyridone compound according to the above (1), which is represented by the formula (3)

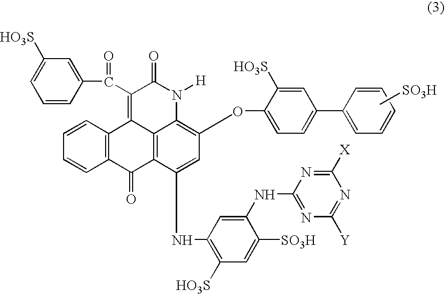

(wherein, X and Y have the same meanings as in the formula (1))
in free acid form,
(4) The anthrapyridone compound according to any one of the above (1) to (3), wherein X is (1) an anilino group which may have a group selected from the group consisting of a sulfonic acid group, a carboxyl group, a methyl group, a methoxy group, an anilino group and a phenoxy group as a substituent; (2) a carboxy-hydroxyanilino group; (3) a phenoxy group which may have a group selected from the group consisting of a sulfonic acid group, a carboxyl group, an acetylamino group, an amino group, a hydroxy group, a phenoxy group and a phenyl group as a substituent, and Y is a hydroxy group; an amino group; a mono- or di-alkylamino group which may have a substituent selected from the group consisting of a sulfonic acid group, a carboxyl group and a hydroxy group on the alkyl group; or a morpholino group,
(5) The anthrapyridone compound according to any one of the above (1) to (3), wherein X is an anilino group which may have a substituent selected from the group consisting of a sulfonic acid group and a carboxyl group; a methyl-sulfoanilino group; a carboxy-sulfoanilino group; a phenoxy group which may be substituted by a carboxyl group, and Y is a hydroxy group or an amino group,
(6) An anthrapyridone compound represented by the following formula (4)

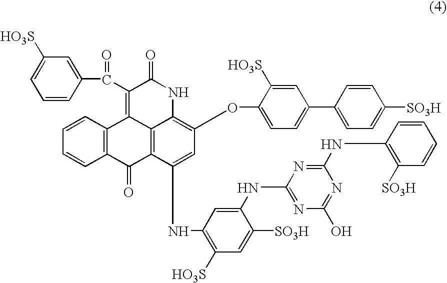

in free acid form,
(7) An anthrapyridone compound represented by the following formula (5)

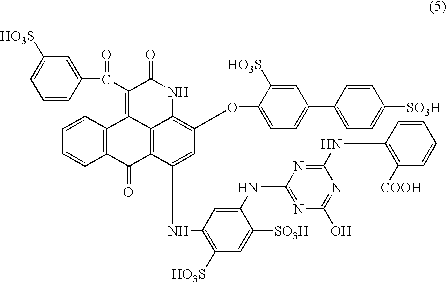

in free acid form,
(8) An ink composition characterized by comprising the anthrapyridone compound according to any one of the above (1) to (7),
(9) The ink composition according to the above (8), which comprises water and a water-soluble organic solvent,
(10) The ink composition according to the above (9), which is for inkjet,
(11) The ink composition according to any one of the above (8) to (10), wherein the content of an inorganic substance in the anthrapyridone compound according to any one of the above (1) to (7) is not more than 1% by weight,
(12) The ink composition according to any one of the above (8) to (11), wherein the content of the anthrapyridone compound according to any one of the above (1) to (7) is 0.1 to 20% by weight,
(13) An inkjet recording method characterized by using the ink composition according to any one of the above (8) to (12) as an ink in an inkjet recording method of performing recording on a record-receiving material by discharging ink droplets responding to a recording signal,
(14) The inkjet recording method according to the above (13), wherein the record-receiving material is a sheet for information transmission,
(15) The inkjet recording method according to the above (14), wherein the sheet for information transmission has an ink image-receiving layer comprising a porous white inorganic substance,
(16) A colored article colored with the ink composition for inkjet recording according to any one of the above (8) to (12),
(17) The colored article according to the above (16), wherein coloring is performed by an ink jet printer,
(18) An ink jet printer loaded with a container comprising the ink composition according to any one of the above (8) to (12),
(19) The anthrapyridone compound according to (1), wherein R₂ is a sulfophenyl group,
(20) An anthrapyridone compound represented by the following formula (7a)

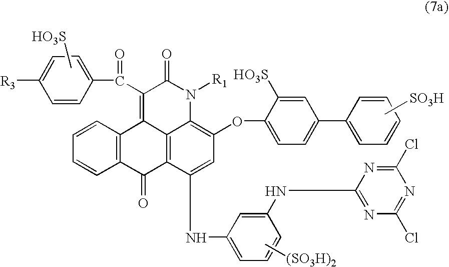

(wherein, R₁ represents a hydrogen atom, an alkyl group, a hydroxy lower alkyl group, a cyclohexyl group, a mono- or di-alkylaminoalkyl group or a cyano lower alkyl group, and R₃ represents a hydrogen atom or a methoxy group, respectively).

### Effect of the Invention

The anthrapyridone compound of the formula (1) of the present invention exhibits a hue of very high vividness and lightness on inkjet recording paper, is excellent in water-solubility, and has a characteristic of good filtration property to membrane filters in the process of producing the ink composition. In addition, the ink composition of the present invention using this compound does not exhibit crystal precipitation, change in physical property and color, or the like after storage for a long period of time, and exhibits good storage stability. And articles printed by using the anthrapyridone compound of the present invention as a magenta ink for inkjet recording and have an ideal magenta hue without selecting a record-receiving material (paper, film and the like). Further, the magenta ink composition of the present invention can make it possible to faithfully reproduce the hue of photo like color images on paper. Furthermore, even when recording is performed on a record-receiving material where inorganic fine particles are coated on the surface, such as paper (film) for inkjet for photo image quality, by using said composition, fastnesses of the recorded article such as light fastness, ozone gas fastness and moisture fastness are good and storage stability of the photo like recorded images for a long period of time is excellent. Accordingly, the anthrapyridone compound of the formula (1) is extremely useful as an ink coloring matter for inkjet recording.

### BEST MODE FOR CARRYING OUT THE INVENTION

The present invention will be specifically explained. In this connection, a sulfonic acid group and a carboxyl group are shown in free acid form unless otherwise specified in the present invention. These groups shown in free acid form may be in the form of salt and the present invention also comprises them.

The coloring matter for ink of the present invention is represented by the above formula (1) in free acid form.

And the lower alkyl group in the present invention can include ones having 1 to 6 carbon atoms, preferably 1 to 4 carbon atoms, and more preferably a methyl group, an ethyl group or a propyl group, among the above alkyl groups. The same goes for the term “lower” which is used other than lower alkyl groups, for example, lower alcohols, in the present invention. In addition, for example, the anilino group which may have a group selected from the group consisting of a sulfonic acid group, a carboxyl group, a methyl group, a methoxy group, an anilino group and a phenoxy group as a substituent can have one or more of the same or different substituents. The same goes for the other similar expressions or expressions with the same effect. Further, in the expressions for a sulfoanilino group, a carboxyanilino group and the like, the sulfonic acid group or the carboxyl group can be one or plural. Furthermore, in the expressions of a carboxy-sulfoanilino group and the like, it is meant basically that one carboxyl group and one sulfo group (sulfonic acid group) are respectively substituted on the benzene ring of anilino group, and the other similar expressions are the same as the above.

In the formula (1), R₁ represents a hydrogen atom, an alkyl group, a hydroxy lower alkyl group, a cyclohexyl group, a mono- or di-alkylaminoalkyl group or a cyano lower alkyl group. The alkyl group in the present invention includes, for example, an alkyl group having 1 to 8 carbon atoms and the like, such as a methyl group, an ethyl group, an n-propyl group, an iso-propyl group, an n-butyl group, a tert-butyl group,' an n-hexyl group and an n-octyl group.

And the hydroxy lower alkyl group in R₁ includes, for example, a hydroxyethyl group, a hydroxypropyl group and the like; the mono-alkylaminoalkyl group includes, for example, a methylaminopropyl group, an ethylamino propyl group and the like; the dialkylaminoalkyl group includes, for example, a dimethylamino propyl group, a diethylaminoethyl group and the like; and the cyano lower alkyl group includes, for example, a cyanoethyl group, a cyanopropyl group and the like. Preferable R₁ includes a hydrogen atom, a lower alkyl group, a hydroxy lower alkyl group, more preferably a hydrogen atom and a lower alkyl group (preferably a methyl group), particularly preferably a hydrogen atom.

In the formula (1) R₂ represents hydrogen, an alkyl group, a phenyl group, a sulfophenyl group, a phenoxy group or a sulfophenoxy group.

When R₂ is a sulfophenyl group and a sulfophenoxy group, the substitution position of the sulfo group is preferably the para-position or the ortho-position to the substitution position of R₂ in the formula (1), more preferably the para-position. For example, o- or p-sulfophenyl, o- or p-sulfophenoxy or the like is included, more preferably p-sulfophenyl.

And the alkyl group in R₂ includes, for example, a methyl group, a tert-butyl group, a tert-amyl group and the like.

Preferable R₂ includes an alkyl group, a sulfophenyl group or a sulfophenoxy group, more preferably a sulfophenyl group or a sulfophenoxy group, most preferably a sulfophenyl group, and preferable is a mono-sulfophenyl group among sulfophenyl groups and p-sulfophenyl among mono-sulfophenyl groups.

R₃ is a hydrogen atom or a methoxy group, preferably a hydrogen atom.

Specific examples of the anilino group which may have a group selected from the group consisting of a sulfonic acid group, a carboxyl group, a methyl group, a methoxy group, an anilino group and a phenoxy group as a substituent in X of the formula (1) include, for example, anilino, an alkylanilino group, for example, 2-methylanilino, 2,6-dimethylanilino, 2,6-diethylanilino, 2,6-diisopropylanilino or 2,4,6-trimethylanilino or the like, a sulfoanilino group, for example, 2-sulfoanilino, 3-sulfoanilino, 4-sulfoanilino, 2,5-disulfoanilino or the like, a methoxy-sulfoanilino group , for example, 4-methoxy-2-sulfoanilino or the like, a methyl-sulfoanilino group, for example, 4-methyl-2-sulfoanilino, 2-methyl-4-sulfoanilino or the like, a carboxyanilino group, for example, 2-carboxyanilino, 4-carboxyanilino, 3,5-dicarboxyanilino and the like, a carboxy-sulfoanilino group, for example, 2-carboxy-5-sulfoanilino, 2-carboxy-4-sulfoanilino or the like, an anilino-sulfoanilino group, for example, 4-anilino-3-sulfoanilino or the like, a carboxy-hydroxyanilino group, for example, 3-carboxy-4-hydroxyanilino or the like, and 4-phenoxyanilino or the like; specific examples of the naphthylamino group which may be substituted by a sulfonic acid group, for example, 1-naphthylamino, 4-sulfo-1-naphthylamino, 5-sulfo-1-naphthylamino, 5-sulfo-2-naphthylamino, 6-sulfo-1-naphthylamino, 7-sulfo-1-naphthylamino, 4,8-disulfo-2-naphthylamino, 3,8-disulfo-1-naphthylamino, 3,6-disulfo-1-naphthylamino, 3,6,8-trisulfo-2-naphthylamino, 4,6,8-trisulfo-2-naphthylamino, 3,6,8-trisulfo-1-naphthylamino or the like, specific examples of the mono alkylamino group which may have a group selected from the group consisting of a sulfonic acid group, a carboxyl group or a hydroxy group as a substituent include, for example, methylamino, ethylamino, propylamino, butylamino, 2-ethylhexylamino, 2-sulfoethylamino, 2-carboxyethylamino, 1,2-dicarboxyethylamino, 1,3-dicarboxypropylamino, 2-hydroxyethylamino, cyclohexylamino and the like, specific examples of the dialkylamino group which may have a group selected from the group consisting of a sulfonic acid group, a carboxyl group and a hydroxy group as a substituent include, for example, dimethylamino, diethylamino, dipropylamino, dibutylamino, bis(carboxymethyl)amino, bis(2-hydroxy ethyl)amino and the like, a specific example of the aralkylamino group includes, for example, benzylamino, a specific example of the cycloalkylamino group includes, for example, cyclohexylamino, specific examples of the phenoxy group which may have a group selected from the group consisting of a sulfonic acid group, a carboxyl group, an acetylamino group, an amino group, a hydroxy group, a phenoxy group and a phenyl group as a substituent include, for example, phenoxy, 4-sulfophenoxy, 4-carboxyphenoxy, 3,5-dicarboxyphenoxy, 4-acetylaminophenoxy, 4-aminophenoxy, 4-hydroxyphenoxy, 4-phenoxyphenoxy, 4-(4-carboxyphenoxy)phenoxy, 4-phenylphenoxy and the like, specific examples of the monoalkylaminoalkylamino group include, for example, 2-(N-methylamino)-ethylamino, 3-(N-methylamino)-propylamino, 3-ethylamino-propylamino or the like, and specific examples of the dialkylaminoalkylamino group include, for example, 3-(N,N-diethylamino)propylamino, 2-(N,N-diethylamino)ethylamino or the like. Preferable X is preferably a sulfoanilino group (more specifically, 2-sulfoanilino or 2,5-disulfoanilino), a lower alkyl-sulfoanilino group (more specifically, 4-methyl-2-sulfoanilino or 2-methyl-4-sulfoanilino), a lower, alkoxy-sulfoanilino group (more specifically, 4-methoxy-2-sulfoanilino), a carboxy-sulfoanilino group (more specifically, 2-carboxy-5-sulfoanilino or 2-carboxy-4-sulfoanilino), a carboxyanilino group (more specifically, 2-carboxyanilino or 3,5-dicarboxyanilino) or a carboxyphenoxy group (more specifically, 3,5-dicarboxyphenoxy), further preferable X includes a sulfoanilino group (more specifically, 2-sulfoanilino or 2,5-disulfoanilino), a carboxy-sulfoanilino group (more specifically, 2-carboxy-5-sulfoanilino or 2-carboxy-4-sulfoanilino), a carboxyanilino group (more specifically, 2-carboxy anilino or 3,5-dicarboxy anilino) or a carboxyphenoxy group (more specifically, 3,5-dicarboxyphenoxy), and particularly preferable X includes 2-sulfoanilino, 2-carboxyanilino, 3,5-dicarboxyanilino, 3,5-dicarboxyphenoxy.

Y in the formula (1) includes, chlorine, hydroxy, amino, 2-sulfoethylamino, 2-carboxyethylamino, carboxymethylamino, 1,2-dicarboxyethylamino, 1,3-dicarboxypropylamino, 2-hydroxyethylamino, 3-(N,N-diethylamino)propylamino, 2-(N,N-diethylamino)ethylamino, bis(carboxymethyl)amino, morpholino or the like, preferably hydroxy, amino, 2-sulfoethylamino, 2-carboxyethylamino, carboxymethylamino, 3-(N,N-diethylamino)propylamino, 2-(N,N-diethylamino)ethylamino or bis(carboxymethyl)amino, more preferably a chlorine atom, hydroxy or amino, further preferably hydroxy or amino, and particularly hydroxy.

For preferably combinations of R₁, R₂, R₃, X and Y, for example, R₁ is a hydrogen atom, a lower alkyl group or a hydroxy-substituted lower alkyl group, preferably a hydrogen atom, R₂ is a sulfophenyl group, a sulfophenoxy group or a C1 to C6 alkyl group, preferably a monosulfophenyl group, and further preferably p-sulfophenyl, R₃ is a hydrogen atom or a lower alkoxy group, and preferably a hydrogen atom, X is anilino, a sulfoanilino group (preferably a mono- or di-sulfoanilino group, and more preferably a mono sulfoanilino group), a carboxyanilino group (preferably mono- or di-carboxyanilino group), a carboxy-sulfoanilino group, a lower alkoxyanilino group, a lower alkylanilino group, benzylamino, cyclohexylamino, a lower alkylamino group, an N,N-di-lower alkylamino lower alkylamino group, a sulfonaphthylamino group or a carboxyphenoxy group (preferably mono- or di-carboxyphenoxy group), more preferable X is a sulfoanilino group (preferably, a mono- or di-sulfoanilino group, more preferably, a monosulfoanilino group), a carboxyanilino group (preferably, a mono- or di-carboxyanilino group) or a carboxyphenoxy group (preferably dicarboxyphenoxy group), and Y is a hydroxy group, an amino group, a carboxy or sulfo lower alkylamino group, a mono- or di-alkanolamino group, an N,N-di-lower alkylamino lower alkylamino group or morpholino, preferably a hydroxy group. The combinations of preferable groups or the combinations of preferable groups and more preferable groups are more preferable.

For more specific combinations of the preferable groups, R₁ is a hydrogen atom or a methyl group, R₂ is a sulfophenyl group, R₃ is a hydrogen atom, X is anilino, 2-sulfoanilino, 2,5-disulfoanilino, 4-methoxy-2-sulfoanilino, 2-carboxy-5-sulfoanilino, 2-carboxyanilino, 4-carboxyanilino, 3,5-di carboxyanilino or 3,5-di carboxyphenoxy, more preferably 2-sulfoanilino, 2-carboxyanilino, 3,5-dicarboxyanilino or 3,5-dicarboxyphenoxy, Y is a hydroxy group or an amino group, more preferably a hydroxy group.

Among the above combinations, particularly preferably, R₁ is a hydrogen atom, R₂ is a 4-(4-sulfo)phenyl group, R₃ is a hydrogen atom, X is anilino, 2-sulfoanilino, 2,5-disulfoanilino, 4-methoxy-2-sulfoanilino, 2-carboxy-5-sulfoanilino, 2-carboxyanilino, 4-carboxyanilino, 3,5-dicarboxyanilino and 3,5-dicarboxyphenoxy, Y is a hydroxy group or an amino group, more preferably a hydroxy group.

Preferable specific examples of the compound represented by the formula (1) include, for example, the following ones.

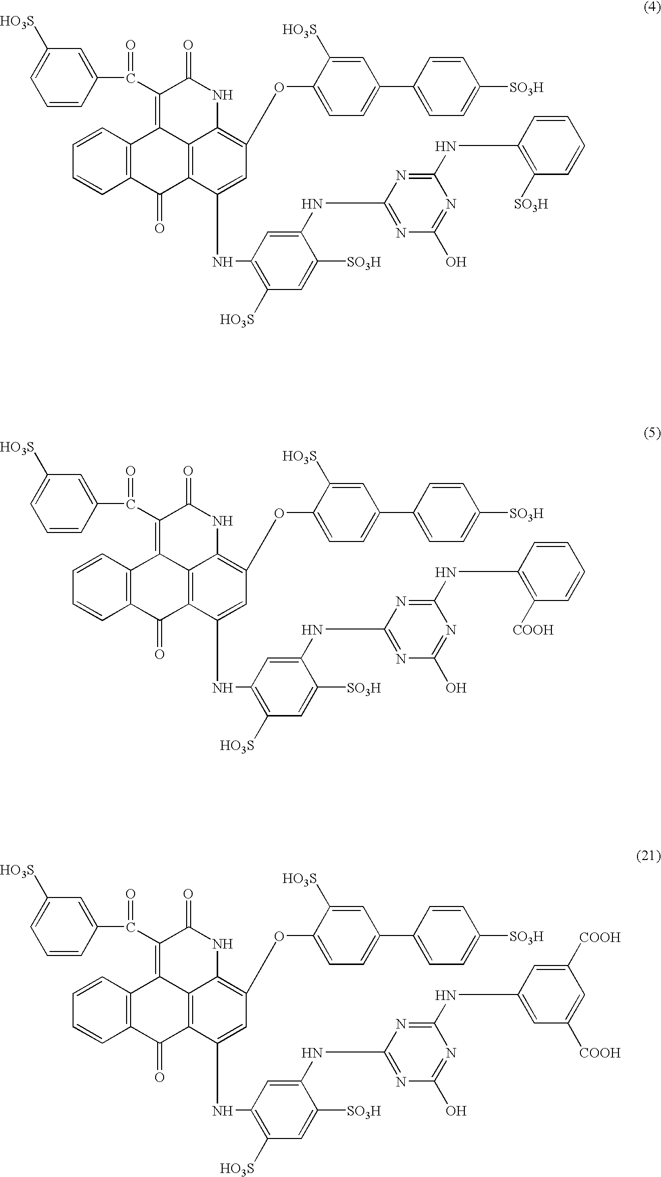

The salts of the compound of the formula (1) include an alkali metal salt, an ammonium salt, a quaternary ammonium salt and the like.

The alkali metal salt is preferably a sodium salt, a potassium salt, a lithium salt or the like. When the quaternary ammonium salt is formed, example of the counter cation is represented by the following formula (24)

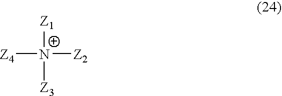

(Wherein, each of Z₁ to Z₄ independently represents a hydrogen atom, an alkyl group, a hydroxyalkyl group or a hydroxyalkoxyalkyl group.).

Examples of the alkyl group in Z₁ to Z₄ of the formula (24) include a methyl group, an ethyl group or the like, examples of the hydroxyalkyl group include a hydroxymethyl group, a 2-hydroxyethyl group, a 3-hydroxypropyl group, a 2-hydroxypropyl group, a 4-hydroxybutyl group, a 3-hydroxybutyl group, 2-hydroxybutyl group or the like, and examples of the hydroxyalkoxyalkyl group include, a hydroxyethoxymethyl group, a 2-hydroxyethoxyethyl group, a 3-(hydroxyethoxy)propyl group, a 3-(hydroxyethoxy)butyl group, a 2-(hydroxyethoxy)butyl group or the like.

Among these salts, preferable salts include a sodium salt, a potassium salt, a lithium salt, a monoethanolamine salt, a diethanolamine salt, a triethanolamine salt, a monoisopropanolamine salt, a diisopropanolamine salt, a triisopropanolamine salt, an ammonium salt and the like. Among them, particularly preferable are salts of lithium, ammonium and sodium.

The above salts can be prepared, for example, by the following method. To a reaction solution containing the compound of the formula (1) (for example, the reaction solution obtained in Example 1 (7) described later in the description) or to a solution dissolving a cake containing the free acid of the formula (1) or its dried one in water, a sodium chloride is added for salting out followed by filtration, to obtain a corresponding sodium salt of the formula (1) as a wet cake. Further, the wet cake is dissolved in water again and the pH is appropriately adjusted by addition of hydrochloric acid to obtain a precipitate, which is then separated by filtration to obtain free acid or a mixture of free acid and a sodium salt in the form of a solid or a wet cake according to need. Furthermore, while stirring the obtained wet cake of free acid or the like of the formula (1) together with water, for example, a potassium hydroxide, a lithium hydroxide, ammonia water, quaternary ammonium of the formula (24) or the like is added to turn it alkaline, to obtain a potassium salt, a lithium salt, an ammonium salt, a quaternary ammonium salt, corresponding to each.

Specific examples of the anthrapyridone compound represented by the formula (2) of the present invention are shown Table 1.

In Table 1, (s) means a sulfonic acid group, (s)2 means a disulfonic acid group, (k) means a carboxyl group, respectively. For example, the notation “2,5-(s)2-anilino” represents anilino substituted by sulfonic acid groups at 2- and 5-positions. In this connection, when R₂ in Table 1 is sulfophenyl, the sulfo group of sulfophenyl is substituted at the para-position to the bonding position of R₂. And OMe represents a methoxy group.

TABLE 1

| Comp. | | | | | |
|---|---|---|---|---|---|
| No. | R₁ | R₂ | R₃ | X | Y |
| 1 | H | 4-sulfophenyl | H | 2-(s)-anilino | OH |
| 2 | H | 4-sulfophenyl | H | 2,5- (s) 2-anilino | OH |
| 3 | H | 4-sulfophenyl | H | 4-(s)-anilino | OH |
| 4 | H | 4-sulfophenyl | H | 3-(s)-anilino | OH |
| 5 | H | 4-sulfophenyl | H | 2-(s)-anilino | NH2 |
| 6 | H | 4-sulfophenyl | H | 2-(s)-anilino | carboxymethylamino |
| 7 | H | 4-sulfophenyl | H | 2-(s)-anilino | carboxyethylamino |
| 8 | H | 4-sulfophenyl | H | 2-(k)-anilino | OH |
| 9 | H | 4-sulfophenyl | H | 4-(k)-anilino | OH |
| 10 | H | 4-sulfophenyl | H | 3-(k)-anilino | OH |
| 11 | H | 4-sulfophenyl | H | 3,5- (k) 2-anilino | OH |
| 12 | H | 4-sulfophenyl | H | 3,5- (k) 2-phenoxy | OH |
| 13 | H | 4-sulfophenyl | H | anilino | OH |
| 14 | H | 4-sulfophenyl | H | benzylamino | OH |
| 15 | H | 4-sulfophenyl | H | cyclohexylamino | OH |
| 16 | H | 4-sulfophenyl | H | n-butylamino | OH |
| 17 | H | 4-sulfophenyl | H | N,N-diethylaminopropylamino | OH |
| 18 | H | 4-sulfophenyl | H | 4-methoxy-2-(s)-anilino | OH |
| 19 | H | 4-sulfophenyl | H | 2-(k)-5-(s)-anilino | OH |
| 20 | H | 4-sulfophenyl | H | 2-(k)-4-(s)-anilino | OH |
| 21 | H | 4-sulfophenyl | H | 4-(s)-naphthyl-1-ylamino | OH |
| 22 | H | 4-sulfophenyl | H | 2-(s)-anilino | monoethanolamino |
| 23 | H | 4-sulfophenyl | H | 2-(s)-anilino | diethanolamino |
| 24 | H | 4-sulfophenyl | H | 2-(s)-anilino | sulfoethylamino |
| 25 | H | 4-sulfophenyl | H | 2-(s)-anilino | N,N-diethylaminopropylamino |
| 26 | H | 4-sulfophenyl | H | 2-(s)-anilino | morpholino |
| 27 | methoxy | 4-sulfophenyl | H | 2-(s)-anilino | OH |
| 28 | H | 4-tert-butyl | H | 2-(s)-anilino | OH |
| 29 | H | 4-methyl | H | 2-(s)-anilino | OH |
| 30 | H | 6-methyl | H | 2-(s)-anilino | OH |
| 31 | H | 4-tert-amyl | H | 2-(s)-anilino | OH |
| 32 | H | 2-sulfophenyl | H | 2-(s)-anilino | OH |
| 33 | H | 4-sulfophenoxy | H | 2-(s)-anilino | OH |
| 34 | methyl | 4-sulfophenyl | H | 2-(s)-anilino | OH |
| 35 | ethyl | 4-sulfophenyl | H | 2-(s)-anilino | OH |
| 36 | 2-hydroxyethyl | 4-sulfophenyl | H | 2-(s)-anilino | OH |
| 37 | H | 4-sulfophenyl | H | 2-methylanilino | OH |
| 38 | H | 4-sulfophenyl | H | 2,6-dimethylanilino | OH |
| 39 | H | 4-sulfophenyl | H | 2,6-diethylanilino | OH |
| 40 | H | 4-sulfophenyl | H | 2,6-diisopropylanilino | OH |
| 41 | H | 4-sulfophenyl | H | 2,4,6-trimethylanilino | OH |

The anthrapyridone compound of the present invention is produced, for example, by the following method. That is, 1 mol of the compound of the following formula (6);

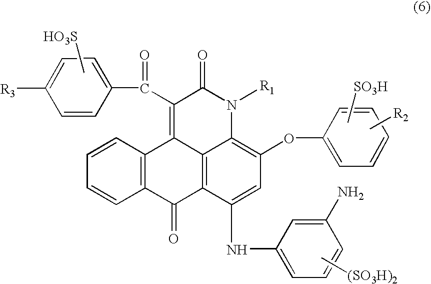

(wherein, R₁, R₂ and R₃ have the same meanings as in the above formula (1)) and 1 to 1.3 mol of 2,4,6-trichloro-1,3,5-triazine(cyanuric chloride) are reacted in water at a pH of 2 to 7 at 5 to 35° C. for 2 to 8 hours to obtain a compound represented by the following formula (7) (a first condensate)

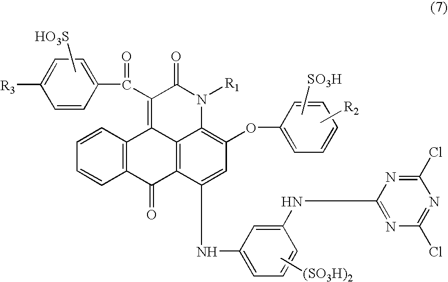

(wherein, R₁, R₂ and R₃ have the same meanings as the above).

With this compound, 1 mol of amines and phenols corresponding to X in the formula (1) is reacted at a pH of 4 to 9 and a temperature of 5 to 90° C. for 10 minutes to 5 hours, to obtain a compound (compound where Y in the formula (1) is a chlorine atom) of the formula (8) (a second condensate)

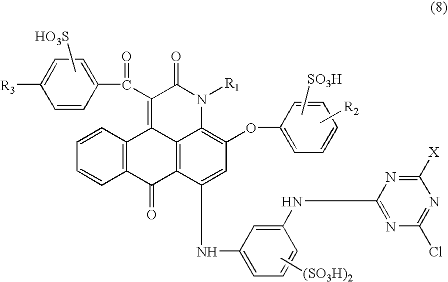

(wherein, R₁, R₂, R₃ and X have the same meanings as in the above formula (1)).

Subsequently, it is hydrolyzed at a pH of 9 to 12 and a temperature of 50 to 100° C. for 10 minutes to 5 hours; or reacted with ammonia or amines corresponding to Y at a pH of 8 to 10 and a temperature of 50 to 100° C. for 10 minutes to 8 hours, to obtain a compound of the formula (9) (a third condensate)

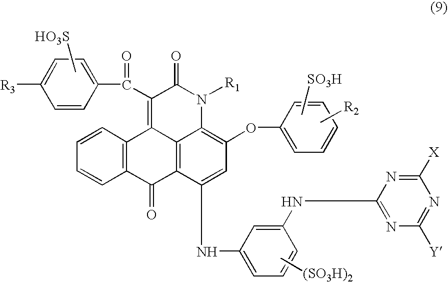

(wherein, R₁, R₂, R₃ and X have the same meanings as the above, Y′ represents a group other than a chlorine atom in Y of the formula (1)).

In this connection, the order of condensation is appropriately determined according to the reactivity of each compound, not limited to the above.

Meanwhile, the anthrapyridone compound represented by the formula (6) can be obtained, for example, by the following way.

That is, to 1 mol of an anthraquinone compound represented by the following formula (10),

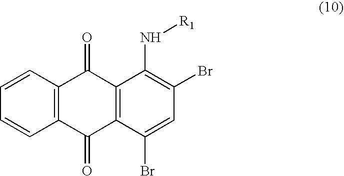

(wherein, R₁ has the same meaning as the above) 1 to 2 mol of phenols represented by the following formula (11)

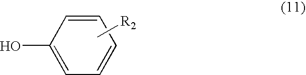

(wherein, R₂ has the same meaning as the above) is reacted in an aprotic polar organic solvent such as N,N-dimethylformamide at 100 to 150° C. for 1 to 8 hours to obtain a compound represented by the following formula (12)

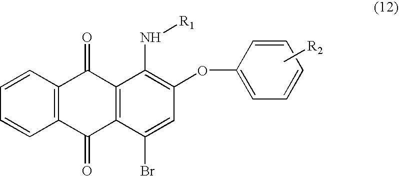

(wherein, R₁ and R₂ have the same meanings as the above).

Subsequently, with 1 mol of a compound represented by the formula (12), under the presence of a base such as a potassium carbonate, 1.1 to 3 mol of substituted or unsubstituted benzoylacetic acid ethyl ester represented by the following formula (13).

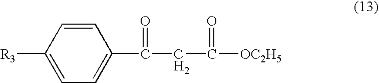

(wherein, R₃ has the same meaning as the above) is reacted in a polar solvent such as xylene under the presence of a basic compound such as a sodium carbonate at 130 to 180° C. for 5 to 15 hours to obtain a compound represented by the following formula (14)

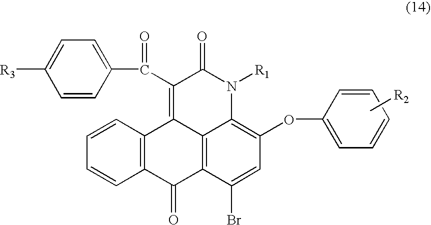

(wherein, R₁, R₂ and R₃ have the same meanings as the above). Subsequently, the obtained compound and 1 to 5 mol of meta-aminoacetoanilide are subjected to Ullmann reaction in an aprotic polar organic solvent such as N,N-dimethylformamide or N-methylpyrolidone, otherwise in an alcohol solvent such as isobutyl alcohol or in a mixed solvent thereof, in the presence of a base such as sodium carbonate or potassium carbonate and a copper catalyst such as copper acetate or copper chloride at 110 to 150° C. for 2 to 15 hours for condensation to obtain a compound represented by the following formula (15)

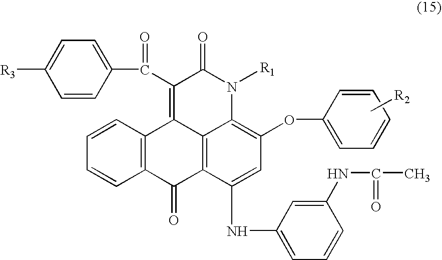

(wherein, R₁, R₂ and R₃ have the same meanings as the above).

Subsequently, the compound represented by the formula (15) is sulfonated and the acetylamino group is hydrolyzed in 8 to 15% by weight of fuming sulfuric acid at 50 to 120° C. to obtain an anthrapyridone compound represented by the following formula (6)

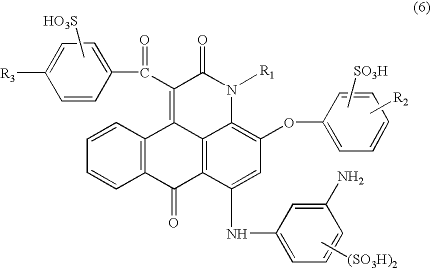

(wherein, R₁, R₂ and R₃ have the same meanings as the above).

The compounds of the present invention are suitable, as magenta coloring matter for ink, for dyeing of natural and synthetic textiles or blends, and these compounds are also suitable for producing inks for writing and ink composition for inkjet recording.

The compound represented by the above formula (1) to be used preferably contains less inorganic substance contained in a whole coloring matter, such as metal cation chloride and, sulfuric acid salt, and the content is, for example, about no more than 1% by weight only as a guide. In order to produce a whole coloring matter having less inorganic substance, for example, a wet cake or its dried one of the anthrapyridone compound of the present invention can be, if necessary, heated and stirred at about 50 to 70° C., in methanol or a methanol-water mixed solvent, filtrated and separated, and if necessary, further washed with methanol and dried, otherwise subjected to desalting treatment by a conventional manner such as a method by a reverse osmosis membrane.

The ink composition of the present invention is provided by dissolving a compound represented by the formula (1) in water or an aqueous solvent (water containing a water-soluble organic solvent described later), and otherwise a reaction solution (for example, such as a reaction solution before addition of 70 parts of a sodium chloride in Example 1 (7) described later) containing a compound represented by the formula (1) of the present invention can be used directly for producing ink compositions. In addition, the object can be isolated from the above reaction solution, dried, for example, spray-dried, and then processed into an ink composition. The ink composition for recording of the present invention typically contains 0.1 to 20% by weight, more preferably 1 to 15% by weight, and further preferably 2 to 10% by weight of the compound of the present invention. The ink composition of the present invention may contain 0 to 30% by weight of a water-soluble organic solvent, 0 to 10% by weight of an ink preparation agent, preferably 1 to 7% by weight, optionally 0 to 5% by weight, respectively. The rest is water.

Specific examples of the water-soluble organic solvent to be used in the present invention include, for example, C1 to C4 alkanol such as methanol, ethanol, n-propanol, isopropanol, n-butanol, isobutanol, secondary butanol or tertiary butanol; carboxylic acid amide such as N,N-dimethylformamide or N,N-dimethylacetamide; lactam such as 2-pyrolidone or N-methyl-2-pyrolidone; cyclic ureas such as 1,3-dimethylimidazolidin-2-one or 1,3-dimethylhexahydropyrimid-2-one; ketone or keto alcohol such as acetone, methylethylketone or 2-methyl-2-hydroxypentan-4-on; cyclic ether such as tetrahydrofuran or dioxane; monomer, oligomer or polyalkylene glycol or thioglycol having a (C2 to C6) alkylene unit such as ethylene glycol, 1,2- or 1,3-propyleneglycol, 1,2- or 1,4-butyleneglycol, 1,6-hexyleneglycol, diethylene glycol, triethylene glycol, tetraethylene glycol, dipropylene glycol, thiodiglycol, polyethylene glycol or polypropylene glycol; polyol (triol) such as glycerine or hexane-1,2,6-triol; polyhydric alcohol (C1 to C4) alkyl ether such as ethylene glycol monomethyl ether or ethylene glycol monoethyl ether, diethylene glycol monomethyl ether or diethylene glycol monoethyl ether, or butylcarbitol or triethylene glycol monomethyl ether or triethylene glycol monoethyl ether; γ-butyrolactone; dimethylsulfoxide; and the like. These water-soluble organic solvents are used alone or in mixture thereof.

Among them, preferable is 2-pyrolidone, N-methyl-2-pyrolidone, mono-, di- or tri-ethylene glycol, dipropylene glycol or butylcarbitol, more preferably 2-pyrolidone, N-methyl-2-pyrolidone, diethylene glycol or butylcarbitol.

Hereinafter, the ink preparation agents which can be used in preparation of the ink composition of the present invention will be explained. Specific examples of the ink preparation agents include, for example, an antiseptic and fungicide, a pH modifier, a chelating agent, a rust-preventive agent, a water-soluble UV absorbing agent, a water-soluble polymer compound, a dye dissolving agent, a surfactant and the like.

The antiseptic and fungicide includes, for example, organic sulfur, organic nitrogen sulfur, organic halides, haloallylsulfone, iodopropargyl, N-haloalkylthio, benzothiazoles, nitrile, pyridines, 8-oxyquinolines, isothiazolines, dithiol, pyridineoxides, nitropropane, organic tin, phenols, quaternary ammonium salt, triazines, thiadiazines, anilide, adamantanes, dithiocarbamate, brominated indanone, benzyl bromoacetates, inorganic salt and the like compounds. The organic halide compound includes, for example, sodium pentachlorophenol, the pyridineoxide compound includes, for example, sodium 2-pyridinethiol-1-oxide, the inorganic salt compound includes, for example, anhydrous sodium acetate, the isothiazoline compound includes, for example, 1,2-benzisothiazolin-3-one, 2-n-octyl-4-isothiazolin-3-one, 5-chloro-2-methyl-4-isothiazolin-3-one, 5-chloro-2-methyl-4-isothiazolin-3-one magnesium chloride, 5-chloro-2-methyl-4-isothiazolin-3-one calcium chloride, 2-methyl-4-isothiazolin-3-one calcium chloride and the like. The other antiseptic and fungicides include sodium sorbate, sodium benzoate and the like.

Any substance can be used as the pH modifier as long as it can control the pH of the ink to be prepared in the range of 8.0 to 11.0 without any adverse effect on the ink to be prepared. The pH modifier includes, for example, alkanolamines such as diethanolamine and triethanolamine; hydroxides of alkali metal such as lithium hydroxide, sodium hydroxide and potassium hydroxide, ammonium hydroxides, carbonates of alkali metal such as lithium carbonate, sodium carbonate and potassium carbonate, and the like.

The chelating agent includes, for example, sodium ethylenediamine tetraacetate, sodium nitrilotriacetate, sodium hydroxyethylethylenediamine triacetate, sodium diethylenetriamine pentaacetate, sodium uracil diacetate and the like.

The rust-preventive agent includes, for example, acid sulfite salts, sodium thiosulfate, ammonium thioglycolate, diisopropylammonium nitrite, pentaerythritol tetranitrate, dicyclohexylammonium nitrite and the like.

The water-soluble UV absorbing agent includes, for example, sulfonated benzophenone, sulfonated benzotriazole or the like.

The water-soluble polymer compound includes, for example, polyvinyl alcohol, cellulose derivatives, polyamines, polyimines and the like.

The dye dissolving agent includes, for example, urea, ε-caprolactam, ethylene carbonate and the like.

The surfactant includes, for example, anionic surfactant, amphoteric surfactant, cationic surfactant, nonionic surfactant and the like. The anionic surfactant includes alkyl sulfocarboxylic acid salt, α-olefin sulfonate, polyoxyethylene alkyl etheracetate, N-acylamino acid and a salt thereof, N-acylmethyltaurine salt, rosin acid soap, castor oil sulfate, lauryl alcohol sulfate salt, alkylphenol type phosphate ester, alkyl type phosphate ester, alkyl allylsulfonate, diethyl sulfosuccinate, diethylhexyl sulfosuccinic acid, dioctyl sulfosuccinate and the like. The cationic surfactant includes 2-vinylpyridine derivatives, poly-4-vinyl pyridine derivatives and the like. The amphoteric surfactant includes lauryldimethylaminoacetic acid betaine, 2-alkyl-N-carboxymethyl-N-hydroxyethylimidazolinium betaine, coconut oil fatty acid amide propyldimethylaminoacetic acid betaine, polyoctylpolyaminoethylglycine and the like, or imidazoline derivatives and the like. The nonionic surfactant includes, ethers such as polyoxyethylene nonylphenyl ether, polyoxyethylene octylphenyl ether, polyoxyethylene dodecylphenyl ether, polyoxyethylene octylphenyl ether, polyoxyethylene oleyl ether, polyoxyethylene lauryl ether and polyoxyethylene alkyl ether; esters such as polyoxyethylene oleic acid, polyoxyethylene oleic acid ester, polyoxyethylene distearate, sorbitan laurate, sorbitan monostearate, sorbitan monooleate, sorbitan sesquioleate, polyoxyethylene monooleate and polyoxyethylene stearate; acetylene glycols such as 2,4,7,9-tetramethyl-5-decyne-4,7-diol, 3,6-dimethyl-4-octyne-3,6-diol and 3,5-dimethyl-1-hexyn-3-ol (for example, Surfynol 104, 82 and 465, OlfineSTG and the like manufactured by Nissin Chemical Industry Co., Ltd.), and the like. These ink preparation agents are used alone or in mixture thereof.

The water-based ink composition of the present invention can be produced by dissolving a compound represented by the formula (1) in water or the above aqueous solvent (water containing a water-soluble organic solvent) together with the above ink preparation agents and the like.

In the above producing process, the order in which the components are dissolved is not particularly limited. A coloring matter may be dissolved in water or the above aqueous solvent and then ink preparation agents may be added thereto and dissolved; or a coloring matter may be dissolved in water and then an aqueous solvent and ink preparation agents may be added thereto and dissolved. And different orders from these may be made, and an aqueous solvent and ink preparation agents may be added to a reaction solution of a coloring matter or a solution subjected to desalting treatment by a reverse osmosis membrane so as to produce an ink composition. The water to be used in producing the ink composition preferably contains less impurity, such as ion-exchanged water or distilled water. In addition, foreign substances may be removed by carrying out microfiltration, if necessary, using a membrane filter, and when the ink composition is used as an ink for ink jet printer, microfiltration is preferably carried out. The pore size of a filter to be used in carrying out microfiltration is typically 1 μm to 0.1 μm, preferably 0.8 μm to 0.2 μm.

The magenta ink compositions containing the water-soluble anthrapyridone compound of the present invention is suitable for impress printing, copying, marking, writing, drafting, stamping or recording, particularly for inkjet recording. In this case, high quality magenta printed matter by impress printing can be obtained which has good fastnesses against water, sun light, ozone and friction. In addition, the compound of the present invention can be formulated further with dyes of yellow, magenta and the like to provide desirable orange color tone or red color tone. Further, it can be used for other color tones, particularly for black color tone.

The colored article of the present invention is articles colored with the above compound of the present invention. The article to be colored is not particularly limited and includes, for example, paper, textile, cloth (cellulose, nylon, wool and the like), leather, substrates for color filters and the like, but not limited thereto. The coloring method includes, for example, printing methods such as dip dyeing, textile printing, screen printing, and a method by an ink jet printer, preferably a method by an ink jet printer.

The record-receiving material (medium) which can be applied to the inkjet recording method of the present invention includes, for example, sheets for information transmission such as paper, film and the like, fiber, leather and the like. The sheet for information transmission is preferably subjected to surface treatment, and more specifically these substrates are preferably provided with an ink receiving layer. The ink receiving layer can be provided on the above substrates, for example, by impregnating or coating a cation polymer, or provided on the surface of the above substrates by coating a porous white inorganic substance which can absorb coloring matter in the ink such as porous silica, aluminasol or special ceramics, together with a hydrophilic polymer such as polyvinyl alcohol or polyvinylpyrrolidone. Such articles as provided with an ink receiving layer are usually called inkjet paper (film) or glossy paper (film), such as Pictorico (manufactured by Asahi Glass Co., Ltd.), Professional Photopaper, Super Photopaper, and Matte Photopaper (all manufactured by Canon Inc.), Photograph Paper (glossy), Photo Matte Paper and Super Fine Glossy Film (all manufactured by SEIKO-EPSON CORPORATION), Premium Plus Photo Paper, Premium Glossy Film and Photo Paper (all manufactured by Hewlet Packard, Japan Ltd.), PhotoLikeQP (manufactured by Konica Minolta, Japan), and the like. In addition, naturally plain paper can be used.

Among them, it is known that discoloration or fading of an image recorded on a record-receiving material where the surface is applied with porous white inorganic substance is proceeded especially further by ozone gas, but a water-based magenta ink composition of the present invention has so superior gas fastness, showing its effect especially in recording on such a record-receiving material.

The porous white inorganic substance to be used for such a purpose is calcium carbonate, kaolin, talc, clay, diatom earth, synthetic amorphous silica, aluminum silicate, magnesium silicate, calcium silicate, aluminium hydroxide, alumina, lithophone, zeolite, barium sulfate, calcium sulfate, titanium dioxide, zinc sulfide, zinc carbonate or the like.

In order to record on a record-receiving material by means of the inkjet recording method of the present invention, for example, a container containing the above ink composition is set on a predefined position in an inkjet printer and recording can be performed on a record-receiving material in a conventional manner. In the inkjet recording method of the present invention, in addition to a yellow ink composition and a cyan ink composition, a green ink composition, an orange ink composition, a blue (or violet) ink composition, the magenta ink composition of the present invention, if required, a black ink composition and the like can be used in combination. Each color ink composition is injected into each container, and the containers, as well as a container containing a water-based magenta ink composition for inkjet recording of the present invention, are set (loaded) in the predefined positions in an inkjet printer and used for recording. The inkjet printer to be used includes, for example, a printer of piezo method utilizing mechanical vibration, a printer of Bubble Jet (registered trademark) method utilizing bubbles generated by heating, and the like.

A water-based magenta ink composition of the present invention is a vivid magenta color, especially has a highly vivid hue on inkjet glossy paper, and provides high fastness of a recorded image. It also gives high safety to humans.

An ink composition according to the present invention doesn't precipitate or separate during storage. In addition, when the ink produced by the present invention is used in inkjet recording, the injector (ink head) is not blocked. An ink of the present invention doesn't change in physical properties even when used under constant recirculation for a relatively long period of time by a continuous inkjet printer or used intermittently by an on-demand inkjet printer.

### Examples

Hereinafter, the present invention will be more specifically explained by Examples. In this connection, “part” and “%” hereinafter in the description are respectively based on mass, unless otherwise specified.

The mass spectrometry was measured using LC-Mass. The instrument used and analysis conditions are as follows.
Apparatus LCT (manufactured by Micromass)
Ionization method ESI
Detection method Nega
HPLC
Apparatus HP1100 (manufactured by HP)
Column YMC-Pack ODS-A(5 μm) 3.0×150 mm (manufactured by YMC)
Column temperature 40° C.
Mobile phase A: 5 mM AcONH4, B: CH3CN
Gradient B conc: 5%-(30 min)-50%
Flow rate 0.25 ml/min
Sample concentration 1000 ppm
Injection volume 5 μl

### Example 1

(1) While stirring, 381.0 parts of a compound (R₁═H) of the above formula (10), 44.6 parts of a compound (R₂=4-phenyl) (4-hydroxybiphenyl) of the above formula (11) and 19.0 parts of pulverized (100 mesh) potassium carbonate were added, in order of the precedence, to 375.0 parts of N,N-dimethylformamide, and the temperature was raised. The reaction was carried out for 3 hours at a temperatUre of 130 to 140° C. The resulting reaction solution was water-cooled and 450 parts of methanol was added thereto, followed by stirring for 30 minutes, and then the reaction product was filtrated, separated and washed with 700 parts of methanol and 300 parts of hot water of 80° C. in order of the preference, followed by drying to obtain 65.9 parts of a compound (R₁═H, R₂=4-phenyl) of the above formula (12) as a brownish yellow crystal.

(2) While stirring, 65.8 parts of a compound (R₁═H, R₂=4-phenyl) of the formula (12), 1.4 parts of potassium carbonate and 67.2 parts of benzoylacetic acid ethyl ester (a compound of the formula (13): R₃═H) were added, in order of the preference, to 120 parts of xylene, and the temperature was raised. The reaction was carried out for 4 hours at a temperature of 142 to 144° C., and in the meantime ethanol and water produced in the reaction were distilled out of the system as an azeotrope with xylene, to complete the reaction. Subsequently, the reaction solution was cooled to 30° C. and 180 parts of methanol was added thereto and stirred for 30 minutes, and then the reaction product was filtrated, separated and washed with 300 parts of methanol, followed by drying to obtain 65.3 parts of a compound (R₁═H, R₂=4-phenyl, R₃═H) of the above formula (14) as a pale yellow needle crystal.

(3) Next, while stirring, 59.8 parts of a compound (R₁═H, R₂=4-phenyl, R₃═H) of the formula (14), 45.0 parts of meta-aminoacetoanilide, 12.0 parts of cupric acetate monohydrate and 15.9 parts of a sodium carbonate were charged, in order of the preference, into 160.0 parts of N,N-dimethylformamide, and the temperature was raised. The reaction was carried out for 5 hours at 120 to 130° C. It was cooled to about 50° C. After 150 parts of methanol was added thereto and stirred for 30 minutes, the reaction product was filtrated, separated and washed with 500 parts of methanol and 400 parts of hot water of 80° C. in order of the preference, followed by drying to obtain 51.7 parts of a compound (R₁═H, R₂=4-phenyl, R₃═H) of the above formula (15) as a light red crystal.
(4) Next, while stirring and water-cooling, 198.5 parts of 32.6% fuming sulfuric acid was added to 161.5 parts of 96.0% sulfuric acid to prepare 360 parts of 10% fuming sulfuric acid. While water-cooling, 51.4 parts of a compound (R₁═H, R₂=4-phenyl, R₃═H) of the formula (15) was added thereto at no more than 50° C., and the temperature was raised. The reaction was carried out for 4 hours at 85 to 90° C. to sulfonate the compound of the formula (15). Next, the resulting reaction solution was added to 600 parts of ice water. Meanwhile, the solution temperature was maintained at no more than 50° C. by appropriate addition of ice together with the reaction solution thereto. Subsequently, water was added thereto so that the fluid volume was 1000 parts, which was filtrated to remove insoluble content. Next, water was added to the mother liquor so that the volume was 1250 parts, and 250 parts of sodium chloride was added thereto at room temperature and stirred for 3 hours to precipitate a crystal, which was filtrated, separated and washed with 300 parts of 20% sodium chloride aqueous solution to obtain 115.0 parts of a wet cake containing 55.7 parts of a compound of the following formula (16)

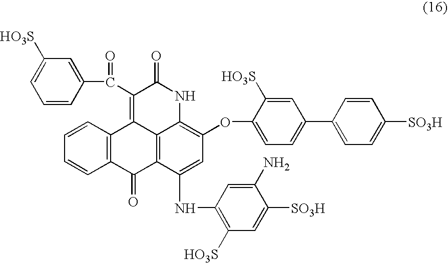

as a red crystal.

The purity of this compound measured by the diazo analysis was 48.4%. Hereinafter, the purities of the other compounds were also measured by the diazo analysis.

1H-NMR (CD3OD, 300 MHz, tetramethylsilane as an internal standard substance): 67.23 (1H, s), 7.32 (1H, d), 7.51 (1H,t), 7.57 (1H, s), 7.59 (1H, t), 7.67 (1H, t), 7.71 (2H, d), 7.81 (1H, dd), 7.91 (2H, d), 7.94 (1H, d), 8.08 (2H, dd), 8.29 (1H, d), 8.31 (1H, s), 8.47 (1H, s) and 8.54 (1H, d).

(5) To 100 parts of water, 106.7 parts of the wet cake (purity: 48.4%) of the compound of the formula (16) obtained in the above (4) was added and then 24 parts of 25% caustic soda was added thereto followed by stirring, and while adjusting the pH at 3 to 4 by further addition of 25% caustic soda, said wet cake was dissolved to obtain a solution containing the compound of the formula (16).

Meanwhile, 0.4 parts of LIPAL OH (trade name, anionic surfactant manufactured by LION CORPORATION) was added to 60 parts of ice water and dissolved, and then 4.4 parts of cyanuric chloride was added thereto and stirred for 30 minutes. The resulting suspension was added in the solution containing the compound of the formula (16) obtained above, and then while adding dropwise a 10% caustic soda aqueous solution to maintain the pH at 2.7 to 3.0, the first condensation reaction was carried out for 3 hours at 25 to 30° C. to obtain a reaction solution containing a compound of the following formula (17).

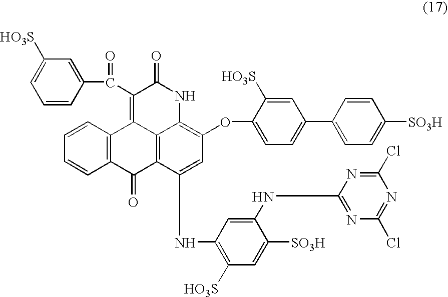

(6) In the reaction solution containing the compound of the formula (17) obtained by the operation of the above (5), a solution consisting of 3.8 parts of orthanilic acid (purity: 98.89%), 20 parts of water and 3.5 parts of 25% caustic soda aqueous solution was added and further water was added thereto so that the volume was 150 parts, and then the temperature was raised. The resulting mixture was maintained at a pH of 6.0 to 6.5 while adding dropwise a 10% caustic soda aqueous solution at a temperature of 60 to 70° C., and reacted for 1 hour for carrying out the second condensation reaction to obtain a reaction solution containing a compound of the following formula (18).

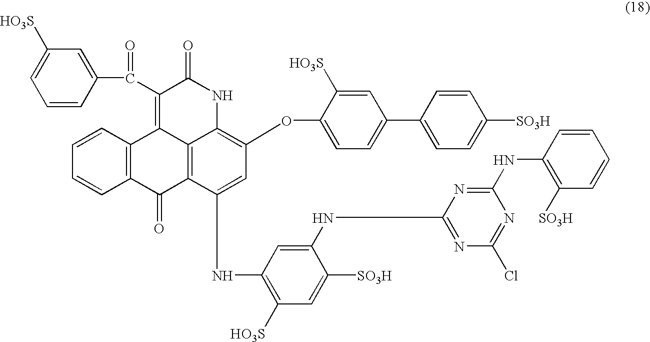

(7) While maintaining the pH at 10.5 to 11.0 by appropriately adding a 25% caustic soda aqueous solution into the reaction solution containing the compound of the formula (18) obtained by the operation of the above (6), the reaction was carried out at a temperature of 85 to 90° C. for 3 hours. After the reaction, water was added to the reaction solution to adjust the fluid volume to 350 parts, followed by filtration to remove insoluble substances.

While maintaining the resulting reaction solution at about 35° C., 70 parts of sodium chloride was added thereto, and then concentrated hydrochloric acid was added thereto to adjust the pH to 0.0, followed by stirring for 1 hour to precipitate a crystal, which was filtrated for separation followed by washing with 200 parts of 20% sodium chloride aqueous solution to obtain 60 parts of a compound of the following formula (4)

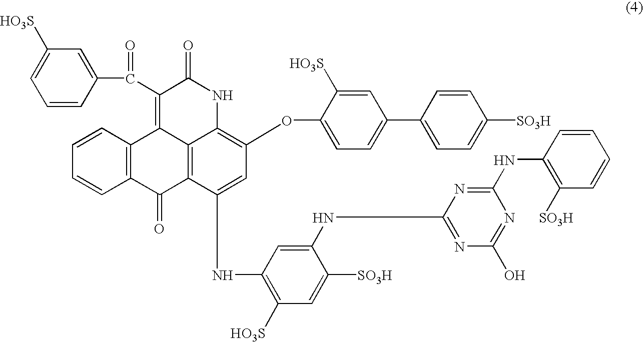

as a red wet cake.

(8) The wet cake obtained by the operation of the above (7) was added in 400 parts of methanol and heated at 60 to 65° C. for 1 hour, and then cooled to room temperature. The crystal was filtrated, washed with methanol and dried to obtain 19.2 parts of a compound (Compound No. 1 in Table 1) represented by the formula (4) as a red crystal (mixture of a sodium salt and free acid). Ions of Cl⁻ and SO₄²⁻ were measured by ion chromatograph and converted to the weight of NaCl and Na₂SO₄ to calculate the inorganic salt content, resulting in no more than 1%.

The maximum absorption wavelength (λmax): 538 nm (in an aqueous solution)

LC-Mass: m/z=1290[M-H+]

### Example 2

(1) By carrying out the operations of (1) to (5) in Example 1, a reaction solution containing a compound represented by the formula (17) was obtained. In said reaction solution, an aqueous solution consisting of 3.2 parts of anthranilic acid, 3.7 parts of 25% caustic soda aqueous solution and 15 parts of water was added, which was reacted for 30 minutes, while adding dropwise a 25% caustic soda aqueous solution at a temperature of 27 to 30° C. to maintain the pH at 4.8 to 5.2, for carrying out the second condensation reaction to obtain a reaction solution containing a compound of the following formula (19).

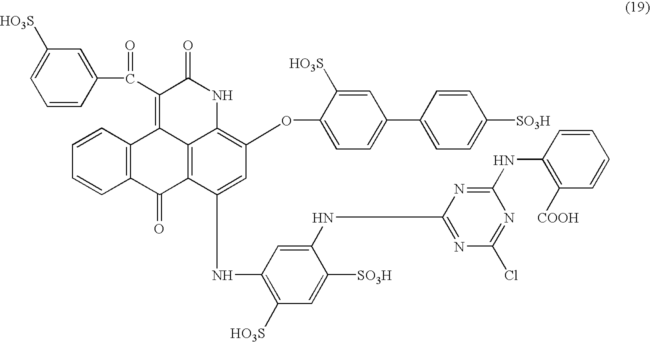

(2) While maintaining the pH at 10.8 to 11.2 by appropriately adding a 25% caustic soda aqueous solution in the reaction solution containing the compound of the formula (19) obtained by the operation of the above (1), the reaction was carried out at a temperature of 90 to 95° C. for 2 hours. After the reaction, water was added to said reaction solution to adjust the fluid volume to 350 parts, followed by filtration to remove insoluble substances. In the resulting reaction solution, 70 parts of sodium chloride was added at room temperature, and then hydrochloric acid was added thereto to adjust the pH to 0.1. The solution was stirred at room temperature for 1 hour to precipitate a crystal, which was filtrated, separated and washed with 100 parts of 20% sodium chloride aqueous solution to obtain 65 parts of a compound of the following formula (5)

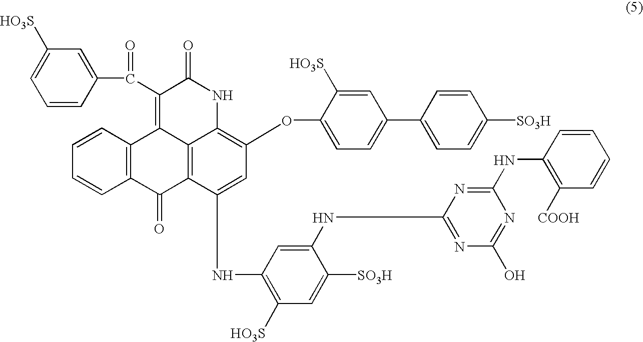

as a red wet cake.

(3) The wet cake obtained by the operation of the above (2) was added in 300 parts of methanol and stirred at room temperature for 1 hour to obtain a crystal, which was filtrated, washed with methanol and dried to obtain 15.0 parts of a compound (Compound No.8 in Table 1) represented by the formula (5) as a red crystal (mixture of a sodium salt and free acid). As a result of measuring in the same manner as in Example 1, the inorganic salt content was not more than 1%.

The maximum absorption wavelength (λmax): 535 nm (in an aqueous solution)

### Example 3

(1) By carrying out the operations of (1) to (5) in Example 1, a reaction solution containing a compound represented by the formula (17) was obtained. In said reaction solution, an aqueous solution consisting of 4.4 parts of 5-amino-isophthalic acid, 7.7 parts of 25% caustic soda aqueous solution and 15 parts of water was added, which was reacted for 30 minutes, while adding dropwise a 25% caustic soda aqueous solution at a temperature of 50 to 60° C. to maintain the pH at 5.8 to 6.2, for carrying out the second condensation reaction to obtain a reaction solution containing a compound of the following formula (20).

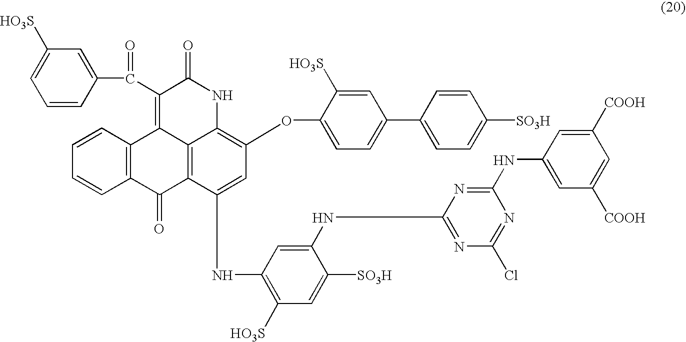

(2) While maintaining the pH at 10.8 to 11.2 by appropriately adding a 25% caustic soda aqueous solution in the reaction solution containing the compound of the formula (20) obtained by the operation of the above (1), the reaction was carried out at a temperature of 90 to 95° C. for 2 hours. After the reaction, water was added to said reaction solution to adjust the fluid volume to 250 parts, followed by filtration to remove insoluble substances. In the resulting reaction solution, hydrochloric acid was added at 30 to 40° C. to adjust the pH to 3 and 37.5 parts of sodium chloride was added thereto, and then hydrochloric acid was added thereto to adjust the pH to 2.0. The solution was stirred at 30 to 40° C. for 1 hour to precipitate a crystal, which was filtrated, separated and washed with 200 parts of 15% sodium chloride aqueous solution to obtain 35 parts of a compound of the following formula (21)

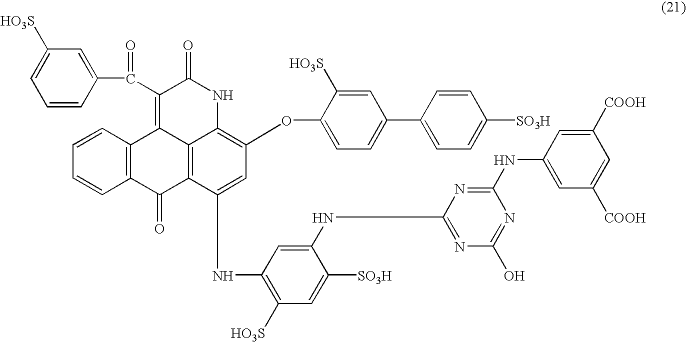

as a red wet cake.

(3) The wet cake obtained by the operation of the above (2) was added in 200 parts of methanol and stirred at 60° C. for 1 hour to obtain a crystal, which was filtrated, washed with methanol and dried to obtain 13.0 parts of a compound (Compound No. 11 in Table 1) represented by the formula (21) as a light red crystal (mixture of a sodium salt and free acid). As a result of measuring in the same manner as in Example 1, the inorganic salt content was not more than 1%.

The maximum absorption wavelength (λmax): 530 nm (in an aqueous solution)

### Example 4

(1) By the operations of (1) to (5) in Example 1, a reaction solution containing a compound represented by the formula (17) was obtained. In it, ice was added to adjust the temperature at 5° C., and then 25% caustic soda was added to adjust the pH to 9.0. An aqueous solution consisting of 4.8 parts of 5-hydroxyisophthalic acid, 8.4 parts of 25% caustic soda aqueous solution and 18 parts of water was added dropwise thereto at 5 to 10° C., while maintaining the pH at 8.8 to 9.2 with 25% caustic soda. After completion of dropwise addition, the solution was heated, and then while adding dropwise a caustic soda aqueous solution at a temperature of 27 to 30° C. to maintain the pH at 8.8 to 9.2, the reaction was carried out for 1 hour for carrying out the second condensation reaction to obtain a reaction solution containing a compound of the following formula (22).

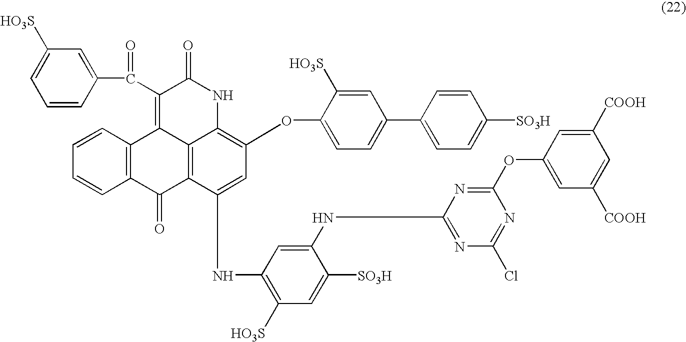

(2) While maintaining the pH at 10.8 to 11.2 by appropriately adding 25% caustic soda aqueous solution in the reaction solution containing the compound of the formula (22) obtained by the operation of the above (1), the reaction was carried out for 2 hours at a temperature of 90 to 95° C. After the reaction, water was added thereto to adjust the fluid volume to 400 parts, followed by filtration to remove insoluble substances. In the resulting reaction solution, 80 parts of sodium chloride was added at room temperature and then hydrochloric acid was added to adjust the pH to 0.0. Said reaction solution was stirred at room temperature for 3 hours to precipitate a crystal, which was filtrated, separated and washed with 100 parts of 20% sodium chloride aqueous solution to obtain 50 parts of a compound of following formula (23)

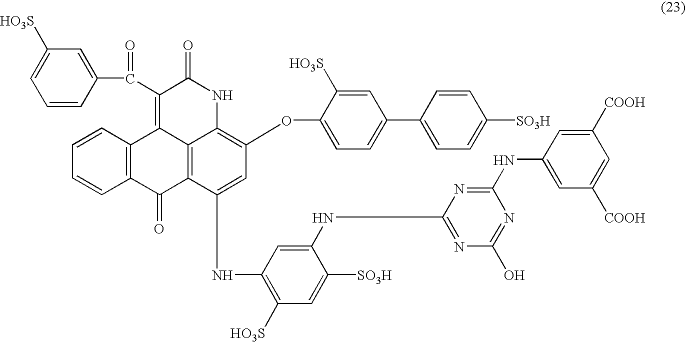

as a red wet cake.

(3) The wet cake obtained by the operation of the above (2) was added in a liquid of 300 parts of methanol and 15 parts of water, heated to 60° C. and stirred for 1 hour to obtain a crystal, which was filtrated, separated washed with methanol and dried to obtain 13.7 parts of a compound represented by the formula (23) as a red crystal (mixture of a sodium salt and free acid). As a result of measuring in the same manner as in Example 1, the inorganic salt content was not more than 1%.

The maximum absorption wavelength (λmax): 531 nm (in an aqueous solution)

### Examples 5 to 8

### (A) Preparation of Ink

The compound (Compound Example No. 1) obtained in the above Example 1 was used to prepare a solution of the composition shown in the following Table 2, which was filtrated with a 0.45 μm membrane filter for removing insoluble matter to obtain each water-based ink composition for inkjet recording. And ion-exchanged water was used as water. In addition, the pH of the ink composition was adjusted to pH=8 to 10 with a 25% caustic soda aqueous solution, and water was further added so that the whole weight was 100 parts. Example 5 is the test using the compound obtained in Example 1.

Similarly, the compounds obtained in Examples 2 to 4 were used to obtain ink compositions. Examples 6 to 8 are the tests using the compounds obtained in Example 2 to 4, respectively.

TABLE 2
(ink composition)

| | | |
|---|---|---|
| Compound in Example 1 (Compound Example No. 1) | 6.0 | parts |
| Glycerine | 5.0 | parts |
| Urea | 5.0 | parts |
| N-methyl-2-pyrolidone | 4.0 | parts |
| Isopropyl alcohol | 3.0 | parts |
| Butylcarbitol | 2.0 | parts |
| Surfynol 104PG50 | 0.1 | part |
| (nonionic surfactant, manufactured by | | |
| Nissin Chemical Industry Co., Ltd.) | | |
| 25% NaOH water + water | 74.9 | parts |
| Total | 100.0 | parts |

### Comparative Example 1

For comparison, the compound of Example 2 in Patent Literature 3 (Compound No.4 in Patent Literature 3) was used to make the same ink composition as the ink composition shown in Table 2, which was used for performing inkjet recording, and the recorded image was evaluated on various items. The compound used in Comparative Example 1 is represented by the following formula (25).

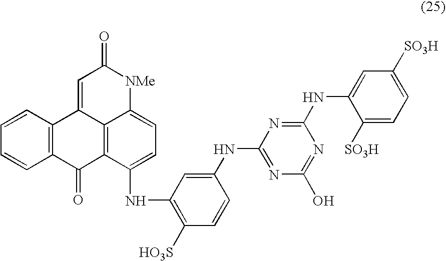

### (B) Inkjet Printing

Using an ink jet printer (manufactured by Canon Inc., trade name: Pixus 860i), inkjet recording was performed on glossy paper (manufactured by Canon Inc., trade name: Professional Photo Paper PR-101) having an ink image-receiving layer containing porous white inorganic substance and photo paper (glossy) manufactured by SEIKO-EPSON CORPORATION. In inkjet recording, an image pattern was made to obtain gradations of several stages in printing density and a print was made.

### (C) Evaluation of Recorded Image

### 1. Evaluation of Hue

### 1-1. Evaluation of Hue on Glossy Paper

For the hue and vividness of recorded image, printed recording paper was measured using a Color Calibration Systems (trade name: GRETAG SPM50, manufactured by GretagMacbeth AG) to calculate values of L*, a* and b*, and chroma (C*) showing vividness was calculated from chromaticity (a*, b*) by C*=((a*)²+(b*)²)^{1/2}. The hue was evaluated by comparing with a sample of standard magenta in Japan Color manufactured by JPMA (Industry Association: Japan Printing Machinery Association).

The results of the hues in Examples 5 to 7 are shown in Table 3. In this connection, paper used for standard magenta in Japan Color was Japan Color Standard Paper.

TABLE 3

| | Lightness | Chromaticity | | Chroma |
|---|---|---|---|---|
| | L* | a* | b* | C* |
| JNC standard magenta | 46.3 | 74.4 | −4.8 | 74.6 |

Glossy paper manufactured by Canon Inc.

| | | | | |
|---|---|---|---|---|
| Example 5 (No. 1) | 57.8 | 88.2 | −2.9 | 88.2 |
| Example 6 (No. 8) | 55.4 | 82.1 | 0.5 | 82.1 |
| Example 7 (No. 11) | 54.8 | 78.7 | 1.0 | 78.6 |
| Example 8 (No. 12) | 53.2 | 82.9 | −1.4 | 83.0 |
| Comparative Example 1 | 49.8 | 81.0 | −6.7 | 81.3 |

Glossy paper manufactured by SEIKO-EPSON CORPORATION

| | | | | |
|---|---|---|---|---|
| Example 5 (No. 1) | 56.1 | 82.3 | −1.7 | 82.3 |
| Example 6 (No. 8) | 56.1 | 80.6 | −2.2 | 80.5 |
| Example 7 (No. 11) | 57.2 | 77.4 | −2.6 | 77.6 |
| Example 8 (No. 12) | 51.9 | 83.2 | −7.2 | 83.5 |
| Comparative Example 1 | 49.5 | 79.9 | −9.5 | 80.4 |

As is cleared from Table 3, in comparison of chromaticity value a*, the values in Examples 5 to 8 are 78.7 to 88.2 and the value in Comparative Example 1 is 81.0 when glossy paper manufactured by Canon Inc. was used, and the values in Examples 5 to 8 are 77.4 to 83.2 and the value in Comparative Example 1 is 79.9 when glossy paper manufactured by SEIKO-EPSON CORPORATION was used, resulting in that the both papers show the almost same values and the values are near to 74.4 of JNC standard magenta.

Further in chromaticity value b*, the values in Examples 5 to 8 are −2.9 to 1.0 and the value in Comparative Example 1 is −6.7 when glossy paper manufactured by Canon Inc. was used, and the values in Examples 5 to 8 are −7.2 to −1.7 and the value in Comparative Example 1 is −9.5 when glossy paper manufactured by SEIKO-EPSON CORPORATION was used, resulting in that there is not so big difference observed between the both papers and the values are near to −4.8 of JNC standard magenta.

In comparison of chroma value C* showing vividness, the values in Examples 5 to 8 are 78.6 to 88.2 and the value in Comparative Example 1 is 81.3 when glossy paper manufactured by Canon Inc. was used, and the values in Examples 5 to 8 are 77.6 to 83.5 and the value in Comparative Example 1 is 80.4 when glossy paper manufactured by SEIKO-EPSON CORPORATION was used, resulting in that the both paper show values higher than 74.6 of the value C* of JNC standard magenta but there is not clear difference observed in comparison of Examples 5 to 8 and Comparative Example 1.

In comparison of lightness value L*, however, the values in Examples 5 to 8 are 53.2 to 57.8 and the value in Comparative Example 1 is 49.8 when glossy paper manufactured by Canon Inc. was used, and the values in Examples 5 to 8 are 51.9 to 57.2 and the value in Comparative Example 1 is 49.5 when glossy paper manufactured by SEIKO-EPSON CORPORATION was used, and further, the value of JNC standard magenta is 46.3, resulting in that it is found that recorded images with the ink composition using the coloring matter of the present invention clearly show values higher than those of Comparative Example 1 and JNC standard magenta and have very high lightness.

As is clear from the above results, it is found that the recorded images of the ink composition using the coloring matter of the present invention has characteristics that it exhibits the same hue as that of Comparative Example 1, and chroma higher than that of JNC standard magenta, and lightness highly superior to those of Comparative Example 1 and JNC standard magenta. Accordingly, it can be said that the anthrapyridone compound of the present invention is suitable as a magenta coloring matter for inkjet.

Hereinafter, the evaluation for fastnesses will be described. Evaluation for fastness is conducted by testing on three items, light fastness, ozone gas fastness and moisture fastness.

### (D) Xenon Light Fastness Test of Recorded Image

Using a xenon weatherometer Ci4000 (manufactured by ATLAS Electric Devices Co.), pieces of printed testing paper of glossy paper manufactured by Canon, Inc. and glossy paper manufactured by SEIKO-EPSON CORPORATION, on which an air layer was provided and a glass plate having a thickness of 2 mm was set up, were irradiated for 50 hours at an illuminance of 0.36 W/m² under the circumstances of a humidity of 60% RH and a temperature of 24° C. Color difference (ΔE) before and after the test was measured and the evaluation was performed on 3 levels.

$$Δ E ≦ 10 …o$$

$$10 < Δ E ≦ 20 …△ \\ Δ E > 20 … \times$$

The results are shown in Table 4.

### (E) Ozone Gas Fastness Test of Recorded Image

Using an ozone weatherometer (manufactured by Suga Test Instruments Co., Ltd.), pieces of printed testing paper of glossy paper manufactured by Canon, Inc. and glossy paper manufactured by SEIKO-EPSON CORPORATION were left for 3 hours under the circumstances of an ozone concentration of 12 ppm, a humidity of 60% RH and a temperature of 24° C. Color difference (ΔE) before and after the test was measured and evaluation was performed on 3 levels.

$$Δ E ≦ 10 …o$$

$$10 < Δ E ≦ 20 …△$$

$$Δ E > 20 … \times$$

The results are shown in Table 4.

### (F) Moisture Fastness Test of Recorded Image

Using a thermo-hygrostat (manufactured by OHKEN, Co., LTD), pieces of printed testing paper of glossy paper manufactured by Canon, Inc. and glossy paper manufactured by SEIKO-EPSON CORPORATION were left for 168 hours at a temperature of 30° C. and a humidity of 85% RH. Bleed property before and after the test was determined by visual observation and evaluated on 3 levels.

∘: Bleed is not observed.

Δ: Bleed is observed on small degree.

×: Bleed is observed on large degree.

The results are shown in Table 4.

TABLE 4

| | | |
|---|---|---|
| | Ozone gas | Moisture |
| Light fastness | fastness | fastness |

Glossy paper manufactured by CANON INC.

| | | | |
|---|---|---|---|
| Example 5 (No. 1) | ∘ | ∘ | ∘ |
| Example 6 (No. 8) | ∘ | ∘ | ∘ |
| Example 7 (No. 11) | ∘ | ∘ | ∘ |
| Example 8 (No. 12) | ∘ | ∘ | ∘ |
| Comparative Example 1 | Δ | x | ∘ |

Glossy paper manufactured by SEIKO-EPSON CORPORATION.

| | | | |
|---|---|---|---|
| Example 5 (No. 1) | ∘ | ∘ | ∘ |
| Example 6 (No. 8) | ∘ | ∘ | ∘ |
| Example 7 (No. 11) | ∘ | ∘ | ∘ |
| Example 8 (No. 12) | ∘ | ∘ | ∘ |
| Comparative Example 1 | Δ | x | ∘ |

As is clear from Table 4, it is obvious that even when any of glossy papers manufactured by CANON INC. and manufactured by SEIKO-EPSON CORPORATION., is used, the recorded image in Comparative Example 1 has a problem in light fastness and a drawback in ozone gas fastness. As compared to this, the recorded image using the ink composition containing the anthrapyridone compound of the present invention exhibited high stability on any test of light fastness, ozone gas fastness and moisture fastness.

Judging from the above, it is obvious that the anthrapyridone compound of the present invention is a coloring matter providing images having fastnesses, and it is said that the anthrapyridone compound of the present invention is extremely excellent as a magenta coloring matter for inkjet in this regard.

1. An anthrapyridone compound represented by the following formula (1) 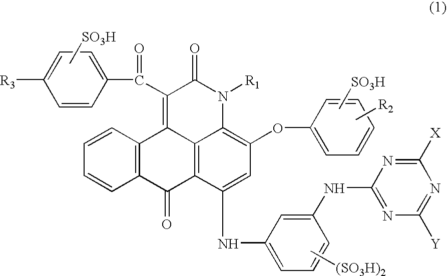
(wherein R₁ represents a hydrogen atom, an alkyl group, a hydroxy lower alkyl group, a cyclohexyl group, a mono- or di-alkylaminoalkyl group or a cyano lower alkyl group, R₂ represents a hydrogen atom, an alkyl group, a phenyl group, a sulfophenyl group, a phenoxy group or a sulfophenoxy group, R₃ represents a hydrogen atom or a methoxy group, X represents (1) an anilino group which may have a group selected from the group consisting of a sulfonic acid group, a carboxyl group, a methyl group, a methoxy group, an anilino group and a phenoxy group as a substituent; (2) a carboxy-hydroxyanilino group; (3) a naphthylamino group which may be substituted by a sulfonic acid group; (4) a mono- or di-alkylamino group which may have a group selected from the group consisting of a sulfonic acid group, a carboxyl group and a hydroxy group as a substituent; (5) an aralkyl amino group; (6) a cycloalkylamino group; (7) a phenoxy group which may have a group selected from the group consisting of a sulfonic acid group, a carboxyl group, an acetylamino group, an amino group, a hydroxy group, a phenoxy group and a phenyl group as a substituent; (8) a monoalkylaminoalkylamino group; (9) a dialkylaminoalkylamino group; (10) a hydroxy group; or (11)an amino group, Y represents a chlorine atom; a hydroxy group; an amino group; a mono- or di-alkylamino group which may have a substituent selected from the group consisting of a sulfonic acid group, a carboxyl group and a hydroxy group on the alkyl group; or a morpholino group, respectively)
in free acid form.

2. The anthrapyridone compound according to claim 1, which is represented by the following formula (2) 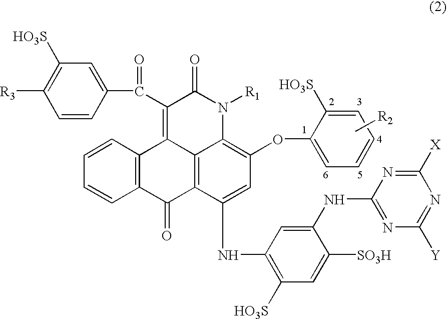
(wherein, R₁, R₂, R₃, X and Y have the same meanings as in the formula (1))
in free acid form.

3. The anthrapyridone compound according to claim 1, which is represented by the formula (3) 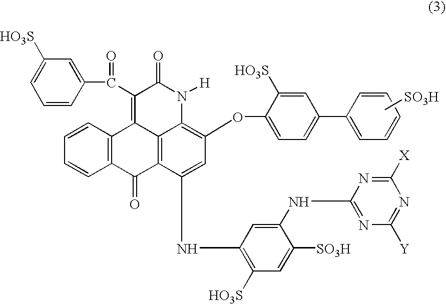
(wherein, X and Y have the same meanings as in the formula (1))
in free acid form.

4. The anthrapyridone compound according to any one of claims 1 to 3, wherein X is (1) an anilino group which may have a substituent selected form the group consisting of a sulfonic acid group, a carboxyl group, a methyl group, a methoxy group, an anilino group and a phenoxy group; (2) a carboxy-hydroxyanilino group; (3) a phenoxy group which may have a substituent selected from the group consisting of a sulfonic acid group, a carboxyl group, an acetylamino group, an amino group, a hydroxy group, a phenoxy group and a phenyl group, and Y is a hydroxy group; an amino group; a mono- or di-alkylamino group which may have a substituent selected from the group consisting of a sulfonic acid group, a carboxyl group and a hydroxy group on the alkyl group; or a morpholino group.

5. The anthrapyridone compound according to any one of claims 1 to 3, wherein X is an anilino group which may have a substituent selected from the group consisting of a sulfonic acid group and a carboxyl group; a methyl-sulfoanilino group; a carboxy-sulfoanilino group; a phenoxy group which may be substituted by a carboxyl group, and Y is a hydroxy group or an amino group.

6. An anthrapyridone compound represented by the following formula (4) 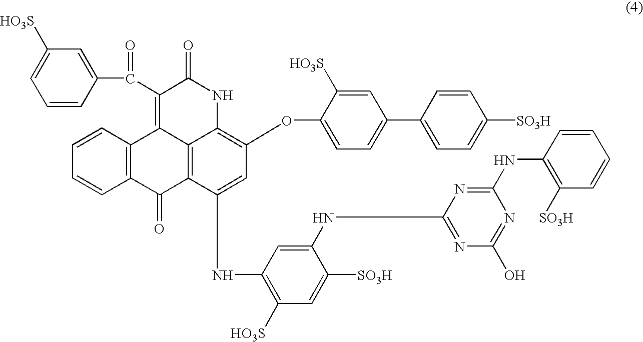
in free acid form.

7. An anthrapyridone compound represented by the following formula (5) 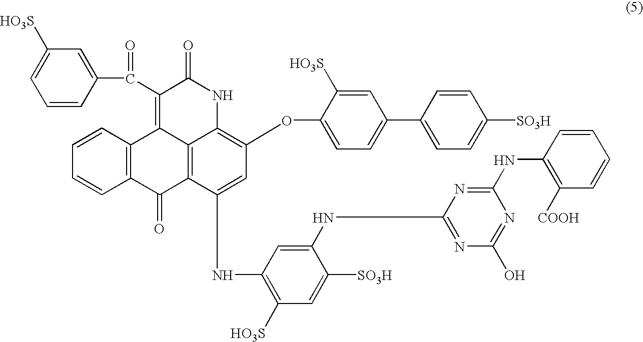
in free acid form.

8. An ink composition characterized by comprising the anthrapyridone compound according to any one of claims 1 to 7.

9. The ink composition according to claim 8, which comprises water and a water-soluble organic solvent.

10. The ink composition according to claim 9, which is for inkjet.

11. The ink composition according to claim 8, wherein the content of an inorganic substance in the anthrapyridone compound according to any one of claims 1 to 7 is not more than 1% by weight.

12. The ink composition according to claim 8, wherein the content of the anthrapyridone compound according to any one of claims 1 to 7 is 0.1 to 20% by weight.

13. An inkjet recording method characterized by using the ink composition according to claim 8 as an ink in an inkjet recording method of performing recording on a record-receiving material by discharging ink droplets responding to a recording signal.

14. The inkjet recording method according to claim 13, wherein the record-receiving material is a sheet for information transmission.

15. The inkjet recording method according to claim 14, wherein the sheet for information transmission has an ink image-receiving layer comprising a porous white inorganic substance.

16. A colored article colored with the ink composition for inkjet recording according to claim 8.

17. The colored article according to claim 16, wherein coloring is performed by an ink jet printer.

18. An ink jet printer loaded with a container comprising the ink composition according to claim 8.

19. The anthrapyridone compound according to claim 1, wherein R₂ is a sulfophenyl group.

20. An anthrapyridone compound represented by the following formula (7a) 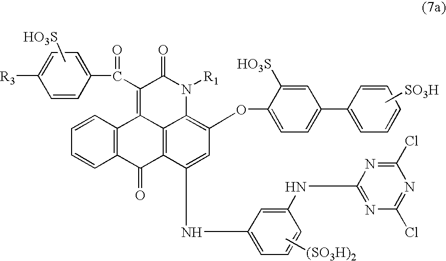
(wherein, R₁ represents a hydrogen atom, an alkyl group, a hydroxy lower alkyl group, a cyclohexyl group, a mono- or di-alkylaminoalkyl group or a cyano lower alkyl group, and R₃ represents a hydrogen atom or a methoxy group, respectively).
